(19) United States
(12) Patent Application Publication
(10) Pub. No.: US 2008/0274196 A1
(43) Pub. Date: Nov. 6, 2008

(54) Oral Pharmaceutical Suspension Compositions Of Fexofenadine

(75) Inventors: Surya Kumar Jayanthi, Maharashtra (IN); Nitin S. Deshmukh, Maharashtra (IN); Himadri Sen, Maharashtra (IN)

Correspondence Address: MERCHANT & GOULD PC, P.O. BOX 2903, MINNEAPOLIS, MN, 55402-0903, US

(73) Assignee: LIPIN LIMITED, Mumbai Maharashtra (IN)

(21) Appl. No.: 11/997,954

(22) PCT Filed: Aug. 3, 2006
(86) PCT No.: PCT/IN06/00277

§ 371 Date: Jun. 6, 2008

(30) Foreign Application Priority Data
Aug. 5, 2005 (IN) 703/KOL/2005

(51) Int. Cl. A61K 31/445; A61K 9/14; A61P 37/08

(52) U.S. Cl. 424/489; 514/317

(57) ABSTRACT

An oral, pharmaceutical suspension composition of Fexofenadine. Fexofenadine is a mixture of compacted Fexofenadine and plain fexofenadine in a ratio of 0.01:0.99 to 0.99 to 0.01 having a mean particle size of fexofenadine particles in the range of 10μ and 250 μ.An oral, pharmaceutical suspension composition of Fexofenadine, which is bioequivalent to a tablet dosage form of fexofenadine marketed under the trade name of Allegra®. Bioequivalence between a suspension formulation and the commercially tablet formulation of fexofenadine i.e. ‘Allegra®’ is achieved by the use of a mixture of compacted Fexofenadine.

### FIELD OF THE INVENTION

The present invention concerns oral, pharmaceutical suspension compositions of Fexofenadine. The invention also concerns a process for the preparation of said suspension compositions of Fexofenadine and the use of said suspension compositions in patient populations including pediatric populations.

### BACKGROUND OF THE INVENTION

Fexofenadine is a well-known synthetic antiallergenic with the chemical name (±)-4-[1-hydroxy-4- [4(hydroxydiphenylmethyl)-1-piperidinyl]-butyl]- α, α -dimethyl benzeneacetic acid. It is a metabolite of terfenadine and an antihistamine with selective peripheral H1-receptor antagonist activity.

Fexofenadine is known from U.S. Pat. No. 4,254,129. It is highly active via oral administration. It is commercially available, in particular as an oral tablet or capsule, under the trade name Allegra®. Allegra® is indicated for the treatment of seasonal allergic rhinitis and uncomplicated skin manifestations of chronic idiopathic urticaria in adults and children 6 years or older. While the capsules are formulated to contain 60 mg of Fexofenadine, the tablets contain 30, 60, or 180 mg fexofenadine hydrochloride.

While numerous pharmaceutical compositions for oral administration have been proposed, there still exists a need for commercially acceptable fexofenadine formulation for oral administration with good patient convenience and acceptance, especially for children or the elderly since these patient populations experience difficulties swallowing the tablets, even with liquids. However, it is known that suspension formulations have a higher bioavailability than solid oral dosage forms. Moreover, bioequivalence between such formulations and the existing commercially available dosage forms of Fexofenadine is of utmost importance.

We have now surprisingly discovered that Fexofenadine can be formulated in the form of suspension compositions, which are bioequivalent to the commercially available formulations of Fexofenadine. Further, we have also surprisingly found that these compositions can be made bioequivalent by careful manipulation of the particle size and structure of the drug particles thus circumventing the need for use of bioavailability enhancing substances such as p-glycoprotein inhibitors such as those disclosed in U.S. Pat No. 6,451,815. Such formulations are envisaged to fulfill the existing need of patient friendly dosage forms especially for the pediatric and geriatric patient populations.

### OBJECT OF THE INVENTION

An object of the invention is to provide an oral, pharmaceutical suspension composition of Fexofenadine.

Another object of the invention is to provide an oral, pharmaceutical suspension composition of Fexofenadine, which is bioequivalent to a tablet dosage form of fexofenadine marketed under the trade name of Allegra®.

Yet another object of the present invention is to provide an oral suspension formulation of Fexofenadine which is bioequivalent to the commercially available fexofenadine tablet formulation, marketed under the brand name ‘Allegra®’ and which exhibit a mean AUC₀₋ₜ in the range of 300 to 800 hr*ng/ml.

Yet another object of the present invention is to provide an oral suspension formulation of Fexofenadine which is bioequivalent to the commercially available fexofenadine tablet formulation, marketed under the brand name ‘Allegrao®’ and which exhibit a mean AUC_{0-inf} in the range of 300-800 hr*ng/ml.

Yet another object of the present invention is to provide an oral suspension formulation of Fexofenadine, which is bioequivalent to the commercially available fexofenadine tablet formulation, marketed under the brand name ‘Allegra®’ and which exhibit a mean Cₘₐₓ in the range of 70-200 ng/ml.

Yet another object of the invention is to provide an oral, pharmaceutical suspension composition comprising of compacted Fexofenadine and plain Fexofenadine, which is bioequivalent to the commercially available fexofenadine tablet formulation, marketed under the brand name ‘Allegrao®’.

Yet another object of the invention is to provide a pharmaceutical composition comprising mixture of fexofenadine as such and compacted fexofenadine particles having a particle size such that the mean particle size of fexofenadine particles are in the range of 10μ and 250μ which when administered in humans demonstrates Cₘₐₓ for fexofenadine which is substantially equivalent to the Cₘₐₓ obtained when an equivalent dose of an oral tablet formulation comprising fexofenadine marketed under the name of “Allegra®” is administered to humans.

### DETAILED DESCRIPTION OF THE INVENTION

The present invention concerns oral, pharmaceutical suspension compositions of Fexofenadine. The suspension dosage form is capable of masking the taste of the drug and also provide the drug in as suitable a form as is possible to dissolve it as fast as possible with maximum patient compliance especially for children and the elderly.

The term “Fexofenadine” is meant to cover Fexofenadine in the form of its racemate or a single enantiomer, in free base form or in acid addition salt form of the racemate or one of its single enantiomers. An acid addition salt form may be prepared from the free base form in a conventional manner and vice-versa. Examples of suitable acid addition salt forms include hydrochloride, lactate and ascorbate. Fexofenadine in the form of a hydrochloride salt is preferred.

The term “suspension composition” includes within its scope but is not limited to compositions selected from the group consisting of pellets for suspension which can be coated or uncoated, granules for suspension, in the form of a unit dose packet (sometimes referred to in the art as a “sachet”), in the form of a suspension made from a unit dose packet, in the form of a powder for oral suspension, in the form of a dose sipping device and in the form of an oral suspension per se (liquid suspension) and combinations of these e.g. coated pellets filled in a dose sipping device or in a sachet. It is noted that when a unit dose packet is constituted, it is probably mainly in the form of a suspension if reconstituted according to directions, although the extent of suspension versus solution depends on a number of factors such as pH. The use of the term “suspension” herein is intended to embrace liquids containing fexofenadine partially in suspension and partially in solution.

Preferred oral, pharmaceutical suspension compositions comprising Fexofenadine are in the form of powder for suspension and in the form of a liquid suspension.

For the purposes of the present invention, Fexofenadine can be used as such or can be milled, micronized or can be compacted (e.g. by roller compaction or by slugging in a tablet compression machine) prior to use. Fexofenadine obtained by such different processes can also be combined in one composition. In a preferred embodiment, Fexofenadine as such is combined with compacted Fexofenadine in a ratio ranging from 0.01 to 0.99 to 0.99 to 0.01. In a further preferred embodiment, Fexofenadine as such is combined with compacted Fexofenadine in a ratio ranging from 0.2 to 0.8 to 0.8 to 0.2. In a still preferred embodiment, Fexofenadine as such is combined with compacted Fexofenadine in a ratio ranging from 0.3 to 0.7 to 0.7 to 0.3.

The mixture of fexofenadine as such combined with compacted fexofenadine in the above ratio has a mean particle size of fexofenadine particles in the range of 10μ and 250μ. This particle size can be achieved by methods commonly known to those skilled in the art and can include methods like dry milling, wet milling, controlled crystallization and micronization.

The term mean particle size as used herein means that “50% particles are between 10μ and 2501μ” and can also be represented as d₅₀ of the fexofenadine particles being between 10μ and 250μ. It is noted that the notation dₓ means that X % of particles have a diameter less than the specified diameter D.

The particle size of the fexofenadine particles is measured for the purpose of this invention using light scattering technique (Malvern Mastersizer Hydro 2000S).

The oral, pharmaceutical suspension composition can additionally comprise of at least one excipient depending upon the dosage form e.g. whether as pellets or as granules for suspension and so on. The excipient can be one or more selected from the group consisting of diluents, binders, disintegrants, stabilizers, wetting agents, sweeteners, thickening agents, dispersing agents, pH—stabilizing agents, flavoring agents, taste—enhancing agents, preservatives, coloring agents, anti-foaming agents, lubricants and flow-aids and the like. One excipient can perform more than one function.

Diluents, which include, but are not limited to mannitol, sucrose, starch, lactose, dicalcium phosphate, xylitol, sorbitol, micro-crystalline cellulose and the like can be used.

Binders which include, but are not limited to, alkylcelluloses such as methyl cellulose, hydroxyalkylcelluloses such as hydroxypropylcellulose, low substituted hydroxypropylcellulose and hydroxypropyl methylcellulose, sodium carboxymethylcellulose or mixtures thereof, pregelatinised maize starch or polyvinylpyrrolidone can be used.

Disintegrants, which include but are not limited to, crospovidone, sodium starch glycolate, starches such as maize starch and dried starch, croscarmellose sodium and cellulose products such as microcrystalline cellulose, microfine cellulose, low substituted hydroxypropylcellulose and the like.

A stabilizer such as an organic acid can also be used. The organic acid can be citric acid, tartaric acid and the like.

Suitable wetting agents can include, but are not limited to, surfactants, either singly or in admixture. Examples of surfactants include, but are not limited to, the polysorbates, sodium lauryl sulphate, poloxamers and the like.

Suitable sweeteners include, but are not limited to, natural sweeteners such as sugars e.g. fructose, glucose, sucrose, sugar alcohols such as mannitol, sorbitol or mixtures thereof and artificial sweeteners such as sodium saccharine, sodium cyclamate and aspartame.

Suitable thickening agents function as suspending agents and include, but are not limited to, hydrocolloid gums known for such purpose, examples of which include xanthan gum, guar gum, locust bean gum, gum tragacanth, microcrystalline cellulose and carboxymethylcellulose sodium, sodium carboxymethylcellulose, polyvinylpyrrolidone, hydroxypropylcellulose and the like or mixtures thereof.

Dispersing agents include, but are not limited to, colloidal silicon dioxide and surfactants, wherein the surfactant is used alone or as an admixture with one or more surfactant. Combinations of colloidal silicon dioxide with one or more surfactants can also be used.

The oral, pharmaceutical suspension composition may also contain a pH-stabilizing agent to maintain a desired pH upon reconstitution, as discussed above. The term “pH-stabilizing agent” encompasses buffers and pH-altering agents. Suitable pH-stabilizing agents include tribasic sodium phosphate, anhydrous sodium carbonate, glycine, citric acid and the like or mixtures thereof.

Flavouring agents are well known to persons skilled in the art and include, but are not limited to fruity flavours. Frescofort Flavour Permaseal, Grenadine Flavour Permaseal and Tutti Frutti Flavour or combinations thereof are preferred.

Taste enhancing agents include, but are not limited to, sodium chloride, glycine, citric acid, tartaric acid and the like and mixtures thereof.

Suitable preservatives include, but are not limited to, benzoic acid and sorbic acid and their salts, methyl paraben, butylparaben, propylparaben and the like.

Suitable coloring agents include, but are not limited to, titanium dioxide pigments, lake colours and iron oxide pigments.

Antifoaming agents include, but are not limited to simethicone emulsion and the like.

Lubricants and flow aids such as, but not limited to, talc, magnesium stearate, calcium silicate and colloidal silicon dioxide can also be used.

All these excipients can be used at levels well known to the persons skilled in the art. The oral suspension compositions can be prepared by means well known to those skilled in the art.

For example, the powder for suspension formulations can be prepared by a process comprising the steps of compacting Fexofenadine, and mixing uncompacted Fexofenadine with compacted Fexofenadine and one or more excipients selected from the group consisting of diluents, binders, disintegrants, stabilizers, wetting agents, sweeteners, thickening agents, dispersing agents, pH-stabilizing agents, flavoring agents, taste—enhancing agents, preservatives, coloring agents, lubricants and flow-aids and the like.

Where applicable, the powder for oral suspension can be reconstituted using potable water or using juices such as apple juice, strawberry juice, orange juice or using aerated or carbonated preparations. Alternatively, for the suspension per se, vehicles well known to persons skilled in the art, such as propylene glycol, glycerin, sorbitol, liquid glucose and the like can also be used, at levels well known to the persons skilled in the art, in addition to water.

The application of the invention can be seen by the following, non limiting examples:

### EXAMPLE 1

### Fexofenadine Hydrochloride Suspension 30 mg/5 ml

| S. No | Ingredients | Ingredients % w/v |
|---|---|---|
| 1. | Fexofenadine Hydrochloride | (0.60%) |
| | | (Compacted 0.18%) |
| | | (Plain 0.42%) |
| 2. | Colloidal silicone Dioxide | 0.40 |
| 3. | Sorbitol solution | 10.0 |
| 4. | Glycerin | 25.0 |
| 5. | Sucrose | 40.0 |
| 6. | Sodium methyl paraben | 0.15 |
| 7. | Sodium propyl paraben | 0.05 |
| 8. | Xanthan gum | 0.28 |
| 9. | Tutti fruitty flavour Code | 0.25% V/V |
| 10. | Citric acid monohydrate | qs to adjust pH (0.06%) |
| 11. | Simethicone emulsion 30% | 0.15 |
| 12. | Sunset yellow (FD&C Yellow no. 6) | 0.01 |
| 13. | Purified water | Q.s to 100% |
| 14. | Magnesium Stearate | 0.0018% |

Brief Manufacturing Procedure:
1. Sunset Yellow was dissolved in some quantity of Purified Water and Xanthan Gum was dispersed in this solution. Sorbitol solution and Glycerine were then added to this solution under stirring
2. To an additional quantity of Purified water was dissolved Sodium Methyl Paraben, Sodium Propyl paraben and sucrose. This solution was filtered and added to the Xanthan gum mucilage of Step 1 while stirring continuously.
3. Colloidal silicon dioxide was dispersed in the bulk of Step 2 while stirring continuously.
4. 30% Simethicone emulsion was dispersed in an additional quantity of Purified water and added to the bulk of Step 3.
5. 70% of Fexofenadine was passed through 100 # mesh S.S. Screen and added to the bulk of Step 4 while stirring continuously.
6. Fexofenadine HCl was mixed with Magnesium Stearate and sifted through 40# S.S sieve and compacted using roll Compacter or slugged using tablet compression machine. The resulting compacts or slugs were milled and screened to collect particles passing through 60 # mesh S.S. screen and retained on 100 # mesh S.S. screen. These granules were slowly added to the above bulk under stirring.
7. The volume was made upto 95% of the total volume with Purified water and stirred for 15 minutes.
8. The suspension was passed through 20 # mesh S.S screen.
9. The pH of the suspension was adjusted to a pH of 4.5 to 5.5 using citric acid.

10. Flavour was added to the above while stirring continuously
11. Volume was made up with Purified Water and stirred for 15 minutes.

### EXAMPLE 2

### Fexofenadine Hydrochloride Powder for Suspension 30 mg/5 ml

| | | Qty in gm/20 gms |
|---|---|---|
| | | of powder for |
| Sr. No. | Ingredient | suspension |
| 1 | Fexofenadine Hydrochloride* | 0.30 gms |
| 2 | Magnesium Stearate | 0.003 gms |
| 3 | Purified Water (To be evaporated) | — |
| 4 | Sunset yellow | 0.0075 gms |
| 5 | Xanthan Gum | 0.20 gms |
| 6 | Sodium methyl paraben | 0.075 gms |
| 7 | Sodium propyl paraben | 0.025 gms |
| 8 | Mannitol | 3.0 gms |
| 9 | Sucrose | 15.74 gms |
| 10 | Colloidal Silicone Dioxide | 0.25 gms |
| 11 | Citric Acid Monohydrate | 0.05 gms |
| 12 | Flavour Tutti Fruitty | 0.30 gms |
| 13 | Aspartame | 0.05 gms |
| | TOTAL | 20.0 gms |

*Compacted API:

Procedure for reconstitution: reconstitute 20 gm up to 50 ml with purified water.

Brief Manufacturing Procedure
1. Compacted Fexofenadine HCl was prepared by mixing Magnesium stearate and Fexofenadine hydrochloride, compacting using roll compactor or slugged using tablet compression machine, milling the compacts or slugs using Multi Mill or Oscillating Granulator using 1 mm/0.5 mm S.S. screen. The milled material was sifted through 60# S.S. sieve and material retained on 100 # mesh was collected.
2. Sucrose, xanthan gum, mannitol, methyl paraben sodium, propyl paraben sodium, and Aerosil 200 were sifted through 40 # mesh S.S screen and mixed well with fexofenadine Hydrochloride of Step 1 in suitable mixer.
3. Sunset yellow was dissolved in purified water along with citric acid monohydrate. This solution was added to powder mix and granulated well
4. The granules were dried in suitable drier at 50 to 60° C. and sifted through 40 # mesh. The granules retained on the 40 # mesh S.S. screen were milled in multi mill using 1 mm/0.5 mm screen, sifted through 40 # S.S. screen and mixed with previously sifted granules.
5. Flavour and aspartame were sifted through 40 # mesh S.S. screen and mixed with the above-sifted powder in suitable mixer.

### Bioequivalence Study

A three-way crossover bioequivalence study was carried out using the suspension compositions of Examples 1 and 2 and using Allegra® 30 mg tablets as the reference.

The study was carried out in nine volunteers in fasting conditions. The study was monitored in terms of the AUC and Cₘₐₓ achieved with the test product and reference product. AUC's are areas under serum concentration of Fexofenadine —time curves. Generally, the values for AUC represent a number of values taken from all the subjects in a population and are, therefore, mean values averaged over the entire population. Cₘₐₓ, the observed maximum serum concentration of Fexofenadine is likewise an average value. The 90% confidence intervals for the ratios of the log transformed mean values for Cₘₐₓ and AUC for the test and reference product (T/R ratio) is a measure of the bioequivalence between the test and reference product. Values between 80 and 125% for these intervals indicate bioequivalence as recommended by the US FDA.

Table 1 indicates the results of the study.

TABLE 1
Three way crossover bioequivalence study between suspension
compositions of Examples 1 and 2 and Allegra ® 30 mg tablets

| | | | |
|---|---|---|---|
| | Log | | |
| | Transformed | 90% | |
| | T/R (%) ratio | Confidence | |
| | of least | Interval of log | |
| | square | transformed | Mean plasma |
| Parameter | means | data | concentration |

Results for Fasting Study for Suspension (Example 1)

| | | | | |
|---|---|---|---|---|
| Cmax | 110.28 | 88.67-137.15 | 122 | ng/ml |
| AUC₀₋ₜ | 98.22 | 86.84-111.10 | 580 | hr * ng/ml |
| AUC_{0-inf} | 103.51 | 87.42-122.57 | 626 | hr * ng/ml |

Results for Fasting Study for Powder for Suspension (Example 2)

| | | | | |
|---|---|---|---|---|
| Cmax | 103.09 | 86.14-123.38 | 110 | ng/ml |
| AUC₀₋ₜ | 96.41 | 82.95-112.06 | 560 | hr * ng/ml |
| AUC_{0-inf} | 97.28 | 82.41-114.84 | 573 | hr * ng/ml |

The results indicate that both the suspension and the powder for suspension are bioequivalent to Allegra® tablets.

While the foregoing specification teaches the principles of the present invention, with examples provided for the purpose of illustration, it will be understood that the practice of the invention encompasses all of the casual variations, adaptations, modifications, deletions or additions of procedures and protocols described herein, as come within the scope of the following claims and its equivalents.

1. An oral, pharmaceutical suspension composition of Fexofenadine.

2. The suspension composition of claim 1 wherein the Fexofenadine is a mixture of compacted Fexofenadine and plain fexofenadine in a ratio of 0.01:0.99 to 0.99 to 0.01 having a mean particle size of fexofenadine particles in the range of 10μ and 250μ.

3. The suspension composition of claim 1 wherein the suspension composition is selected from the group comprising of uncoated pellets for suspension, coated pellets for suspension, uncoated granules for suspension, coated granules for suspension, in the form of a unit dose packet, in the form of a suspension made from a unit dose packet, in the form of a dose sipping device and in the form of an oral suspension per se and combinations of these.

4. The suspension composition of claim 1 is powder for oral suspension selected from the group comprising of uncoated pellets for suspension, coated pellets for suspension, uncoated granules for suspension, coated granules for suspension, in the form of a unit dose packet, in the form of a suspension made from a unit dose packet, in the form of a dose sipping device and in the form of an oral suspension per se and combinations of these.

5. An oral, pharmaceutical suspension composition of Fexofenadine, which is bioequivalent to a tablet dosage form of fexofenadine marketed under the trade name of Allegra®.

6. A method of achieving bioequivalence between a suspension formulation and the commercially tablet formulation of fexofenadine i.e. ‘Allegra®’ by use of a mixture of compacted Fexofenadine and plain fexofenadine wherein the mixture has a ratio of compacted Fexofenadine to plain fexofenadine of 0.01:0.99 to 0.99 to 0.01 and having a mean particle size of fexofenadine particles in the range of 10μ and 250μ.

7. The method of claim 6 wherein the mixture has a ratio of compacted Fexofenadine to plain fexofenadine of 0.30:0.70 to 0.70:0.30.

8. An oral, pharmaceutical suspension composition comprising of compacted Fexofenadine and plain Fexofenadine, which is bioequivalent to the commercially available fexofenadine tablet formulation, marketed under the brand name ‘Allegra®’.

9. A oral suspension formulation of Fexofenadine which is bioequivalent to the commercially available fexofenadine tablet formulation, marketed under the brand name ‘Allegrao®’ and which exhibits a mean AUC₀₋ₜ between 300 to 800 hr*ng/ml.

10. A oral suspension formulation of Fexofenadine, which is bioequivalent to the commercially available fexofenadine tablet formulation, marketed under the brand name ‘Allegra®’ and which exhibits a mean Cₘₐₓ between 70 to 200 ng/ml.

11. An oral suspension formulation of Fexofenadine which is bioequivalent to the commercially available fexofenadine tablet formulation, marketed under the brand name ‘Allegrao®’ and which exhibits a mean AUC_{0-inf} between 300 to 800 hr*ng/ml.
